(19) United States
(12) Patent Application Publication
(10) Pub. No.: US 2008/0018454 A1
(43) Pub. Date: Jan. 24, 2008

(54) REMOTE MOBILE MEDICAL COMMUNICATION APPARATUS, SYSTEM AND METHOD

(76) Inventors: Kuo-Hung Chan, Hsinchu County (TW); Tzeng-Yow Lin, Tainan City (TW); Jiunn-Haur Shaw, Hsinchu City (TW); Wen-Chen Chang, Hsinchu City (TW); John Lin, Hsinchu County (TW); Chun-Jen Lin, Hsinchu City (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, omitted

(21) Appl. No.: 11/309,208

(22) Filed: Jul. 13, 2006

(51) Int. Cl. G08B 1/08; A61B 5/00

(52) U.S. Cl. 340/539.11; 340/539.12; 340/539.13; 600/300

(57) ABSTRACT

A remote mobile medical communication apparatus is suitable for use in communicating with a remote terminal for monitoring and treating a patient. The apparatus comprises a communicating unit, for transmitting a local communication information to the remote terminal, and receiving a remote communication information from the remote terminal. A patient-monitor interface unit receives at least one patient vital-sign signal from the patient, and then transmits to the remote terminal via the communicating unit. A bi-directional audio/video communication unit is for communicating with the remote terminal with an audio/video information via the communicating unit. An instruction from the remote terminal is instructed if the patient needs a treatment. A system and method use the function of the remote mobile medical communication apparatus.

### BACKGROUND OF THE INVENTION

1. Field of Invention

The present invention relates to medical apparatus. More particularly, the present invention relates to remote mobile medical communication apparatus, system and method.

2. Description of Related Art

In considering the remote communication of medical information, the prior art has no an efficient way to communicate between, for example, a hospital and a remote ambulance.

Even though some conventional technologies have proposed the remote communication, the communication is not convenient. Particularly to an accident location or an ambulance with insufficient equipments and manpower, many emergency operations to the patient may be not proceeded due to short information or expert instruction. The medical center, based on the convention manner, cannot efficiently instruct a remote location to perform the first-add operation for the patient.

Since a sufficient first-add on the patient in time can save the patient a lot. How to efficiently provide the sufficient communication being helpful for pre-diagnoses and pre-treatment is strongly needed to improve.

Even though some conventional technologies have proposed some solutions, there is no one being able to efficiently organize the medical first-add. The convenient and efficient way to provide the first-add to a patient is still strongly needed.

### SUMMARY OF THE INVENTION

The invention provides a remote mobile medical communication apparatus, which is in mobile manner and can be implemented at a place of the event occurring or the ambulance. As a result, a remote center can fully realize the vital sign of the patient and can give the necessary instruction to the local terminal for treating the patient.

The invention also provides a system and a method for remote mobile medical communication, based on the function of the foregoing remote mobile medical communication apparatus, so that the communication between a medical center and a local center of the patient can be efficiently set up.

The invention provides a remote mobile medical communication apparatus, suitable for use in communicating with a remote terminal for monitoring and treating a patient. The apparatus comprises a communicating unit, for transmitting a local communication information to the remote terminal, and receiving a remote communication information from the remote terminal. A patient-monitor interface unit receives at least one patient vital-sign signal from the patient, and then transmits to the remote terminal via the communicating unit. A bi-directional audio/video communication unit is for communicating with the remote terminal with an audio/video information via the communicating unit. An instruction from the remote terminal is instructed if the patient needs a treatment.

A remote mobile medical communication system, suitable for monitoring and treating a patient, comprises a server center, for exchanging any information between a medical center and a local center. A mobile medical communication apparatus is implemented at the local center. The apparatus comprises: a communicating unit, for transmitting a local communication information to the medical center, and receiving a communication information from the medical center; a patient-monitor interface unit, for receiving at least one patient vital-sign signal from the patient, and then transmitting to the medical via the communicating unit; and a bidirectional audio/video communication unit, for communicating with the medical center with an audio/video information via the communicating unit, wherein an instruction from the medical center is instructed if the patient needs a treatment. A displaying and communicating equipment, implemented at the medical center for realizing a vital sign, wherein if the patient needs a treatment, the medical center gives an instruction to the local center.

A displaying and communicating equipment is implemented at the medical center for realizing a vital sign and a locality of a patient. Wherein, if the patient needs an emergent treatment, the medical center gives an instruction to the local center.

The invention also provides a method of medical communication, suitable for monitoring and treating a patient, comprises providing a server center for making an authorized communication connection between a responsible medical center and a local center, wherein a locality of the local center is movable. A video/audio bi-direction communication is set up between the responsible medical center and the local center, via the server. At least one patient vital-sign signal taken from the patient is transmitted by the local center. The least one patient vital-sign signal is received at the responsible medical center. An instruction from the responsible medical center is given to the local center, for treating the patient if the patient needs a treatment.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a block diagram, schematically illustrating a remote mobile medical communication apparatus, according to a preferred embodiment of the invention.

FIG. 2 is a drawing, schematically illustrating a remote mobile medical communication system, according to a preferred embodiment of the invention.

FIG. 3 is a drawing, schematically illustrating the content of communicating information in an integrated image.

FIGS. 4A-4C are data managing processes, schematically illustrating a communication method, according to a preferred embodiment of the invention.

### DESCRIPTION OF THE PREFERRED EMBODIMENTS

The invention provides remote mobile medical communication apparatus, system and method, so that the patient vital-sign signals, the audio communication with necessary instruction, the local image, and the position in an electronic-map (E-map) can be at least managed together in communication. As a result, for example, the pre-diagnoses and the necessary preparation in advance between hospital and ambulance can be proceeded.

In considering a remote large accident locality, such as an earthquake location, so many patients need first-add. The medical equipments and manpower of medical experts are in short. Even if the patients are moved to the ambulance for transporting to the hospital, the situation of the patient cannot be known by the hospital and the ambulance usually has no doctor to provide the first treatment. According to the conventional manner, the patient cannot be actually treated until the patient is indeed transported to the hospital. In considering at least the conventional issues, the present invention can provide the sufficient communication so that the patient can be effectively treated before reaching the hospital.

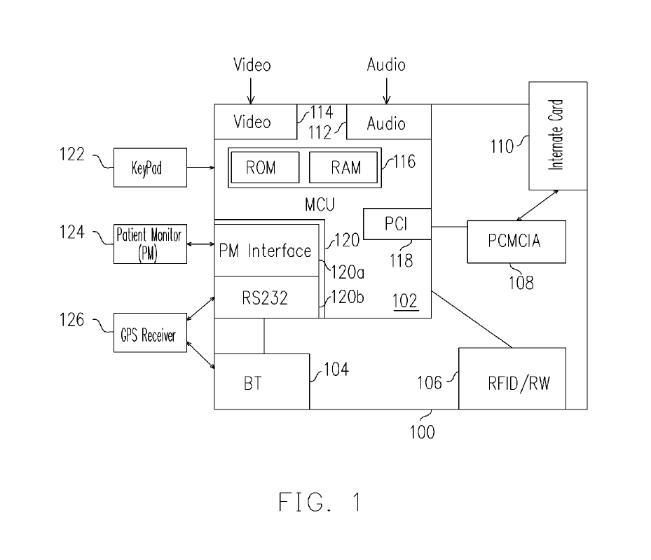

FIG. 1 is a block diagram, schematically illustrating a remote mobile medical communication apparatus, according to a preferred embodiment of the invention. In FIG. 1, the remote mobile medical communication apparatus 100 has the function of fetching the signals from the portable patient monitor, communicating in bi-direction for audio and video information, positioning with electronic map via, i.e. with global position system (GPS), and radio frequency identification (RFID). The communication can be based on network, such as the mobile phone communication.

In better detail, the mobile medical communication apparatus 100 can include a micro-control unit (MCU) 102, a network interface 108 such as the PCMCIA interface connected to an internet card 110, a wireless interface unit 104, such as blue tooth (BT) interface, and a RFID reader 106. The RFID reader 106 can allow the basic information of the patient, which is, for example, stored in the RFID tag and carried by the patient, to be quickly read and identified. In addition, the RFID reader 106 can also have a writing function as a writer. In this situation, for example, the RFID tag for the new patient can be directly input with the basic information, for example, via the keypad. In other word, the RFID reader 106 can be an ID reader/writer unit for reading or optionally writing the basic information with respect to the patient. The MCU 102 can further comprise an audio interface unit 112, a video interface unit 114 for communication with, for example, the remote managing center, a memory unit 116 having i.e. ROM and RAM, a patient monitor interface 120, a data transmission interface 118 for connection with the network interface 108 in communication.

The patient monitor interface 120 can adapt various standards for different type of patient monitors, so that the mobile medical communication apparatus 100 is not limited to a single type of the patient monitor (PM). The signal of patient monitor can be received from the patient monitor interface 120. The patient monitor interface 120 can further include other interfaces for different type of patient monitors. However, a standard interface may be set up by the manufacturers for convenience. Further for example, the GPS receiver 126 can be included for positioning. The GPS receiver can be implemented inside or outside of the medical communication apparatus 100. This GPS information is helpful when the mobile medical communication apparatus 100 is implemented in a moving ambulance, so that the actual location can be known by both the ambulance and the responsible hospital. The interface of GPS receiver 126 usually use the RS232 standard for transmitting data, so that the RS232 interface 120b can be either implemented in the patient monitor interface 120 or directly coupled to the blue tooth in the wireless interface unit 104. The GPS information and the vital-sign signal can be simplified by the MCU 102, so as to reduce the occupation of bandwidth in communication. However, basically, the vital-sign signal can save more bandwidth than the GPS information. The saved bandwidth can be used by, for example, the video interface unit 114 for improving communication efficiency.

It should be also noted that the RS232 is just an example. Some other interfaces, such as the USB, UART (Universal Asynchronous Receiver-Transmitter), I²C (Inter-integrated Circuit), SPI (Serial Peripheral Interface) or LAN interface, can be included too. The memory unit 116 stores all standards of the interfaces for used in the patient monitor interface 120.

Alternatively, all of the connection interfaces and transmission protocols can be arranged into rather a standard interface, which is stored in the memory unit 116. As a result in general, the memory unit 116 can provide the needed protocol for adapting various patient monitors. Then, most of patient monitors can have the property of plug-and-play. The medical communication apparatus 100 is then not limited to adapting a specific patient monitor.

In addition, if it is necessary, a keypad set 122 can be also included for allowing inputting some additional information.

Based on the integrated structure of the patient monitor interface 120, for example, the doctor at the hospital can know the status of the patient including the image and the vital-sign signal, in substantially real time. Then, the person at the ambulance or the accident location can also receive the instruction from the doctor to proceed the proper first add. The GSP information can provide the relative location between the hospital and the ambulance. Then, some preparation may be made beforehand. This can efficiently save the time in emergency operation. The mobile medical communication apparatus 100 indeed can provide the efficient way in mobile property for medical information communication. The medical treatment can then be conveniently proceeded without limitation by the poor information and medical manpower.

The medical communication apparatus 100 can transmit, for example, the PM information, the actual scene of the patient, the doctor instructions, the E-map, and so on. Further, the video apparatus and the audio apparatus may be the external connection to the medical communication apparatus 100. However, the video apparatus and the audio apparatus may also be implemented or mechanically carried by inside the medical communication apparatus 100. When in use, the video apparatus and the audio apparatus can be, for example, pulled out to the proper positions to take image and voice communication. In this situation, the video apparatus and the audio apparatus are always carried with the medical communication apparatus 100. However, the audio interface unit 112 and a video interface unit 114 can adapt other external video apparatus and the audio apparatus when in connection. In other words, medical communication apparatus 100 allows various input sources of video and audio in bi-direction communication. If one video apparatus or the audio apparatus is broken, another one can be used also. Further, since the video apparatus and the audio apparatus are carried by the medical communication apparatus 100, it can assure that at least one video apparatus and the audio apparatus can be used at the event locality.

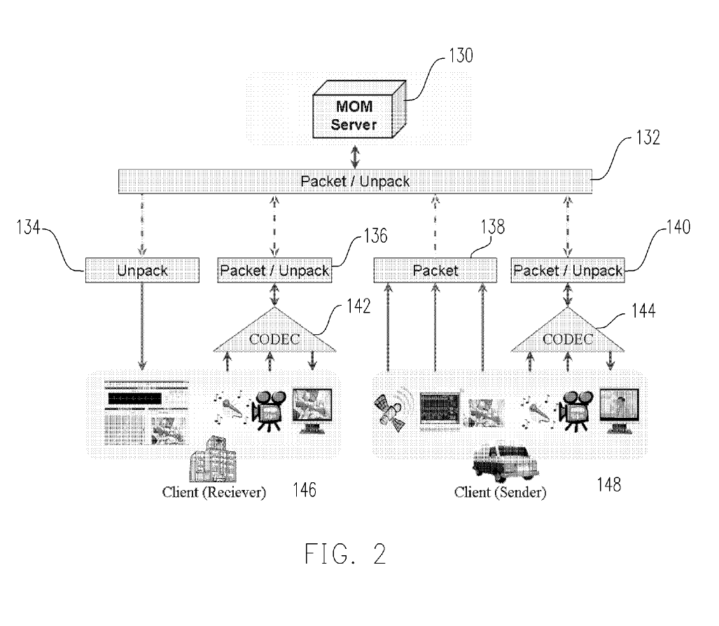

Further, FIG. 2 is a drawing, schematically illustrating a remote mobile medical communication system, according to a preferred embodiment of the invention. In FIG. 2, the communication can use the conventional MOM server 130 to handle the communication. When a communication route is built up, one client, such as a responsible hospital called as a receiver side 146 while the other remote location is called a sender side 148, such as the ambulance. Once the communication connection between the receiver side 146 and the sender side 148, the information can be communicate. At the receiver side 146, a video unit, an audio unit with speaker and microphone, and a displaying terminal can be implemented, so that the hospital can directly see the patient and other information, including the patient vital-sign signal and the location of the ambulance in time. The doctor at the hospital of the receiver side 146 can directly communicate with the persons at the ambulance of the sender side 148, so as provide some instruction to treat the patient. At the sender side 148, similarly, the person at the ambulance can communicate with the doctor in audio and video manner. In addition, the image of the patient can be sent to the hospital. Also, the GPS information is sent, so that the location of the ambulance can be known by the hospital. In other hand, the ambulance can know how far the responsible hospital is.

Here, several conventional hardware units for coding and decoding data as well as packing and unpacking data are necessary, such as the blocks 132-144. Since the function of coding and decoding data as well as packing and unpacking data can be done by any of conventional ways, and should be known by those with ordinary skill of the art, the further descriptions are omitted.

In communication, for example, the transmitting interface of WCDMA, usually used for the mobile phone, can be for example used for transmitting data.

Further in considering the communication bandwidth, usually, the video needs to use a large portion of the bandwidth. In this situation, the patient vital-sign signal and the information received by the GPS receiver can be converted into the information in smaller size, so that the saved bandwidth can be used by, for example, transmitting image information. However, basically, the vital-sign signal can save more bandwidth than the GPS information.

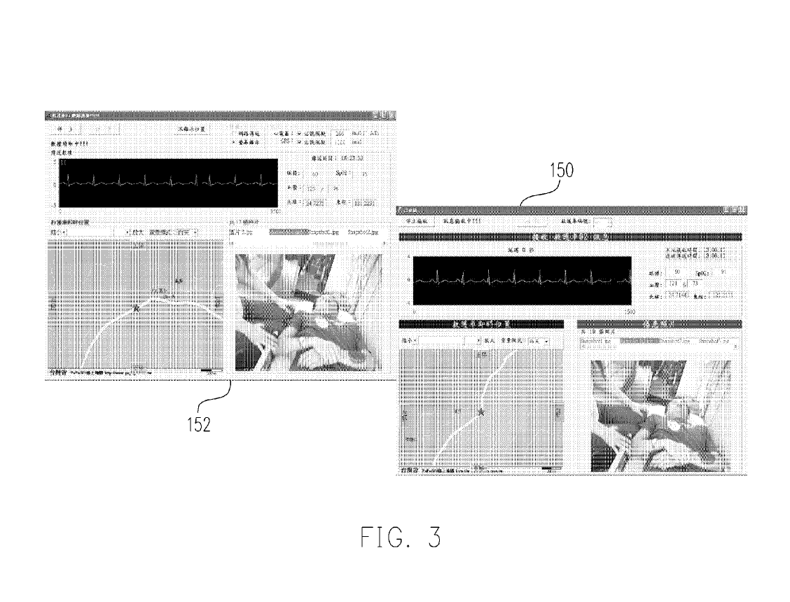

FIG. 3 is a drawing, schematically illustrating the content of communicating information in an integrated image. In FIG. 3, several kinds of information can be integrated in an image display. The images 150, 152 can be, for example, seen at the hospital of the receiver side 146 and the ambulance of the sender side 148. The content of the image can display the actual scene of the patient and the patient vital-sign signal and any other information. However, the content of image is not limited to the example of FIG. 3. The connection status of communication can also be indicated on the image. Therefore, the proper first-add can be instantly proceeded. This is quite helpful for saving the patient.

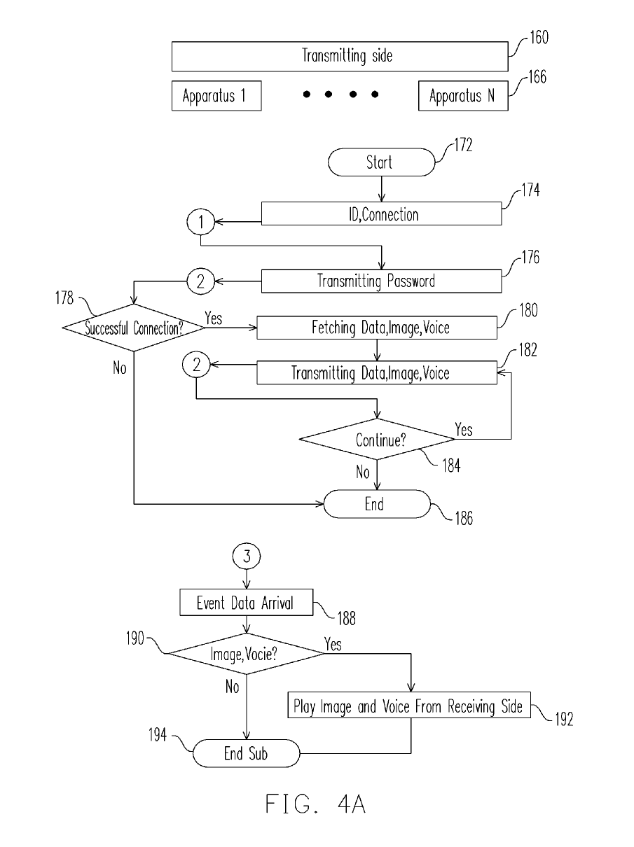

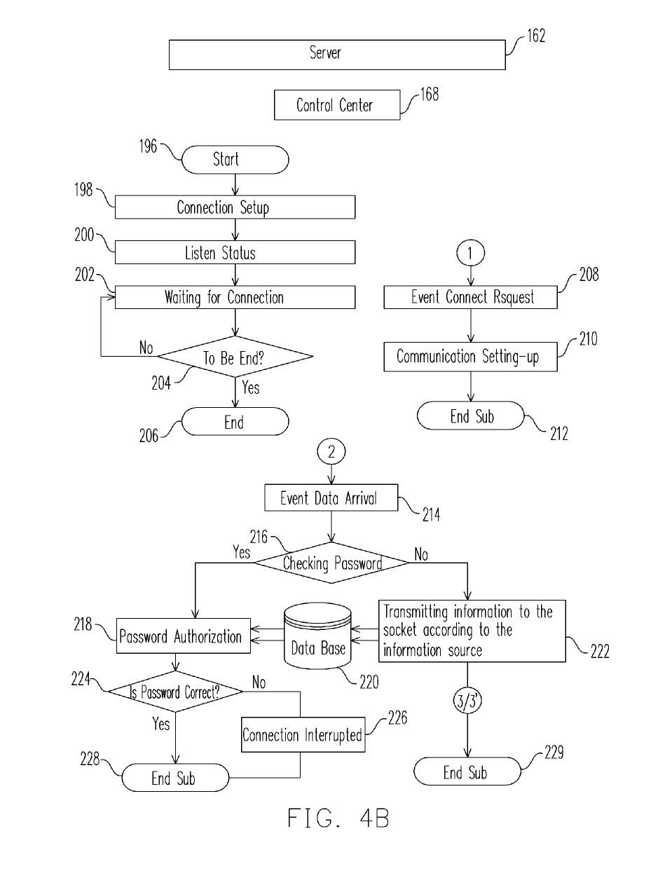

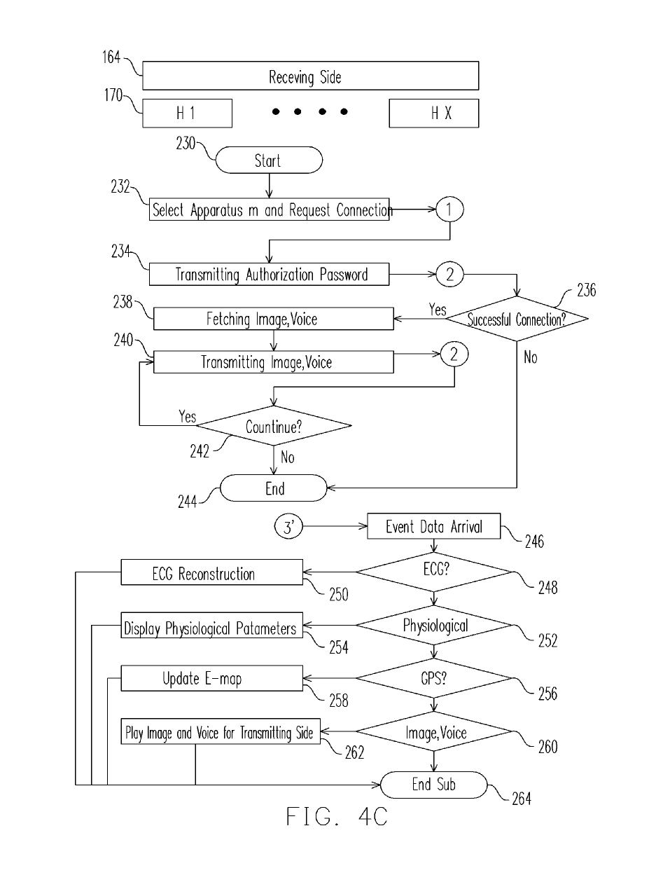

FIGS. 4A-4C are data managing processes, schematically illustrating a communication method, according to a preferred embodiment of the invention. Based on the system in FIG. 2, the data managing processes relating to three sides can be for example shown. The three sides are, for example, named a transmitting side 160, a MOM server side 162, and a receiving side 164. The transmitting side 160 may have at least one mobile apparatuses 166 of FIG. 1, such as Apparatus 1-Apparatus N. The MOM server side 162 may have a control center 168. The receiving side 164 may have, for example, at least one hospital, H1-Hx. The MOS server side may the initial processes in steps 196-206, to setup the sockets for connections under request. At the transmitting side 160, after the step 172 for start, one Apparatus n with an identification (ID) may request a connection, in step 174. The request is sent to (1) at the MOM server side 162. In step 208, the MOM server receives the event connection request. In step 210, communication is set up at an available socket, then in step 212, the subscription for connection ends. In other words, the Apparatus n is connected to the MOM server. However, a responsible hospital m at the receiving side 164 may request the connection to one of the Apparatus, such as the Apparatus n, at the transmitting side, in step 232. Then, the hospital Hm is connected to the Apparatus n through the MOM server.

After the client requests the connection, the MOM server is triggered to set the connection with proper socket, as shown in steps 196-206 of FIG. 4B. Basically, the MOM server keeps on running to monitor any request of connection and is responsible for storing and transferring the data. After the connection is set, the MOM server in the second part under the process (2) can properly manage the data stream to between the transmitting side and the receiving side.

After the connection is set up, the client may inform that the client is at a ready status. Here, the client of sender is, for example, an ambulance. The client sends the individual ID with a representative code to the MOM server. Then, the MOM server is in step 214 at the state of event of “data arrival”. In step 216 to step 228, the MOM server takes the ID of the sender as the password and checks with the databank to verify the password, so that a decision whether to keep connection is made.

Likewise, in step 216 to step 228, here, the client of receiver is, for example, the hospital. The hospital client of receiver sends the individual ID with the ID of the sender to be subscribed to the MOM server to trigger the event of “data arrival”. The MOM server takes the ID of receiver as the password to check with the databank about validity. Then, a decision whether to keep connection is made. In step 226, if the MOM server breaks the connection due to invalid password, the client would get to the “close” state, and the software at the client would be stop in running.

However, in step 216 to step 229, if the connection is on, then the sender client (ambulance) can fetch the vital-sign signals, GPS signal, audio/video signals and transmit to the MOM server at the corresponding gateway, under the process node (3) in FIG. 4A about the steps 188-194. At the same time, under the process node (3′) in FIG. 4C about steps 246-264, the hospital, as the receiver, can get the various signals from the ambulance sender to know the status of patient, and then fetch the image and/or voice signals from the doctors and further then transmitted to the MOM server for transmitting to the ambulance sender.

In other words, when any valid event of “data arrival” is triggered, the MOM server properly transmits the data signals between the hospital receiver and the ambulance sender through the gateway. The process nodes (3) and (3′) respectively obtains and transmits the related information in communication via the MOM server. The communication can be stop when a stop signal is transmitted, for example, from either the sender or the receiver.

It should be noted that, the process steps in FIGS. 4A-4C are just the example. The basic features are the efficient communications between the sender and the receiver via the MOM server. Therefore, various information can be communicated between the sender and the receiver with at least bidirectional voice and image. Then, for example, the first-add operation can be effective done in time without waiting for the patient being transported to the hospital.

In general, the medical communication apparatus 100 of the present invention is used in mobile manner, so that the medical communication apparatus 100 can be freely and conveniently used in various event localities for providing the first-add in time.

The communication can be wireless, based on BT or wireless network, or wire network. The network interface can also, for example, based on TCP/IP protocol. In other words, the communication of data stream can be based on the usual communication networks.

In general, based on the apparatus as described in FIG. 1, a remote mobile medical communication system can be set up, suitable for use in communicating between at least one medical center and at least one local center for monitoring and treating a patient at a place, such as the local center. The system can comprise a server center, for exchanging any information between the medical center and the local center. In addition, a mobile medical communication apparatus is implemented at the local center.

The apparatus, as previous description, can comprise a communicating unit, for transmitting any a local communication information to the medical center, and receiving any a communication information from the medical center; a patient-monitor interface unit, for receiving at least one patient vital-sign signal from the patient, and then transmitting to the medical via the communicating unit; a bi-directional audio/video communication unit, for communicating with the medical center with an audio/video information via the communicating unit, wherein an instruction from the medical center is instructed if the patient needs a local treatment; and an electronic map unit, for positioning a locality and transmitting the locality to the medical center.

Then, a displaying and communicating equipment can be implemented at the medical center for realizing a vital sign and a locality of a patient. Wherein, if the patient needs an emergent treatment, the medical center gives an instruction to the local center.

Alternatively, from the managing method point of view, the invention also disclose the method of medical communication, suitable for communicating between at least one medical center and at least one local center for monitoring and treating a patient at a place, such as the local center. The method can generally comprise providing a server center, for making an authorized communication connection between a responsible medical center and the local center, wherein a locality of the local center is movable. A video/audio bi-direction communication is set up between the responsible medical center and the local center, via the server. At least one patient vital-sign signal taken from the patient is transmitted at the local center. The at least one patient vital-sign signal is received at the responsible medical center. The locality of the local center is received at the responsible medical center, based on an electronic map system, at the responsible medical center. An instruction from the responsible medical center is given to the local center, for treating the patient if the patient needs an emergent treatment.

According to the foregoing descriptions of embodiments, the present invention can generally have several modifications as follows.

According to an embodiment of the apparatus in the invention, the communicating unit can comprise a mobile wireless communication unit or an internet communication unit. Wherein, the communicating unit can comprise a blue tooth communication unit.

According to an embodiment of the apparatus in the invention, the patient-monitor interface unit can comprise a memory unit to store at least one interface information, used to handle various signal types of different patient monitors. Wherein, the at least one interface information for the patient-monitor interface unit comprises interfaces of RS232, USB, UART (Universal Asynchronous Receiver-Transmitter), I²C (Inter-Integrated Circuit), SPI (Serial Peripheral Interface) or LAN.

According to an embodiment of the apparatus in the invention, the patient-monitor interface unit can receive information comprising ECG signal, blood oxygen saturation status, non-invasive blood pressure or heart rate. Also, the electronic map unit can comprise a Global Positioning System (GPS) receiver, for positioning the locality.

According to an embodiment of the apparatus in the invention, the bi-directional audio/video communication unit can receive at least one of a video signal and an audio signal from a video device or an audio device in external connection.

According to an embodiment of the apparatus in the invention, the apparatus can further comprise at least one of a video device and an audio device, which is connected with the bi-directional audio/video communication unit when in operation.

According to an embodiment of the apparatus in the invention, the apparatus can further comprise an ID reader/writer to read or write a basic information in an ID tag, carried by the patient. The ID reader/writer can comprise a radio-frequency identification (RFID) reader/writer. Further, a keypad to input information can be included.

According to an embodiment for the foregoing system, the local center is a place where an event occurs, or an ambulance. Also, the communicating unit can comprise a mobile wireless communication unit or an internet communication unit.

According to an embodiment for the foregoing system, the patient-monitor interface unit can comprise a memory unit to store at least one interface information, used to handle various signal types of different patient monitors.

According to an embodiment for the foregoing system, the bi-directional audio/video communication unit can receive at least one of a video signal and an audio signal from a video device or an audio device in external connection.

According to an embodiment for the foregoing system, the mobile medical communication apparatus further comprises at least one of a video device and an audio device, which is connected with the bidirectional audio/video communication unit when in operation.

Some additional detail arrangements may be further included in the system or the method. However, based on the general features of the invention, the implementation may dependent on the actual designs but still falls in the scope of the invention. It will be apparent to those skilled in the art that various modifications and variations can be made to the structure of the present invention without departing from the scope or spirit of the invention. In view of the foregoing descriptions, it is intended that the present invention covers modifications and variations of this invention if they fall within the scope of the following claims and their equivalents.

What is claimed is:

1. A remote mobile medical communication apparatus, suitable for use in communicating with a remote terminal for monitoring and treating a patient, comprising:
a communicating unit, for transmitting a local communication information to the remote terminal, and receiving a remote communication information from the remote terminal;
a patient-monitor interface unit, for receiving at least one patient vital-sign signal from the patient, and then transmitting to the remote terminal via the communicating unit; and
a bi-directional audio/video communication unit, for communicating with the remote terminal with an audio/video information via the communicating unit, wherein an instruction from the remote terminal is instructed if the patient needs a treatment.

2. The apparatus of claim 1, further comprising an electronic map unit, for positioning a locality and transmitting the locality to the remote terminal.

3. The apparatus of claim 1, wherein the communicating unit comprises a mobile wireless communication unit or an internet communication unit.

4. The apparatus of claim 1, wherein the communicating unit comprises a blue tooth communication unit.

5. The apparatus of claim 1, wherein the patient-monitor interface unit comprises a memory unit to store at least one interface information, used to handle various signal types of different patient monitors.

6. The apparatus of claim 5, wherein the at least one interface information for the patient-monitor interface unit comprises interfaces of RS232, USB, UART (Universal Asynchronous Receiver-Transmitter), I²C (Inter-integrated Circuit), SPI (Serial Peripheral Interface) or LAN.

7. The apparatus of claim 1, wherein the patient-monitor interface unit receiving information comprising ECG signal, blood oxygen saturation status, non-invasive blood pressure or heart rate.

8. The apparatus of claim 2, wherein the electronic map unit comprises a global positioning system (GPS) receiver, for positioning the locality.

9. The apparatus of claim 1, the bi-directional audio/video communication unit receiving at least one of a video signal and an audio signal from a video device or an audio device in external connection.

10. The apparatus of claim 1, further comprising at least one of a video device and an audio device, which is connected with the bidirectional audio/video communication unit when in operation.

11. The apparatus of claim 1, further comprising an ID reader/writer to read or write a basic information in an ID tag, carried by the patient.

12. The apparatus of claim 11, wherein the ID reader/writer comprises a radio-frequency identification (RFID) reader/writer.

13. The apparatus of claim 1, further comprising a keypad to input information.

14. A remote mobile medical communication system, suitable for monitoring and treating a patient, comprising:
a server center, for exchanging any information between a medical center and a local center;
a mobile medical communication apparatus, implemented at the local center, wherein the apparatus comprises:
a communicating unit, for transmitting a local communication information to the medical center, and receiving a communication information from the medical center;
a patient-monitor interface unit, for receiving at least one patient vital-sign signal from the patient, and then transmitting to the medical via the communicating unit; and
a bidirectional audio/video communication unit, for communicating with the medical center with an audio/video information via the communicating unit, wherein an instruction from the medical center is instructed if the patient needs a treatment; and
a displaying and communicating equipment, implemented at the medical center for realizing a vital sign, wherein if the patient needs a treatment, the medical center gives an instruction to the local center.

15. The system of the claim 14, wherein the local center is a place where an event occurs, or an ambulance.

16. The system of the claim 14, wherein the mobile medical communication apparatus further comprising an electronic map unit, for positioning a locality and transmitting the locality to the medical center.

17. The system of the claim 14, wherein the communicating unit comprises a mobile wireless communication unit or an internet communication unit.

18. The system of the claim 14, wherein the patient-monitor interface unit comprises a memory unit to store at least one interface information, used to handle various signal types of different patient monitors.

19. The system of the claim 14, wherein the bidirectional audio/video communication unit receiving at least one of a video signal and an audio signal from a video device or an audio device in external connection.

20. The system of the claim 14, wherein the mobile medical communication apparatus further comprises at least one of a video device and an audio device, which is connected with the bidirectional audio/video communication unit when in operation.

21. A method of medical communication, suitable for monitoring and treating a patient, comprising:
providing a server center, for making an authorized communication connection between a responsible medical center and a local center, wherein a locality of the local center is movable;
setting up a video/audio bi-direction communication between the responsible medical center and the local center, via the server;
transmitting at least one patient vital-sign signal taken from the patient, by the local center;
receiving the least one patient vital-sign signal, at the responsible medical center; and
giving an instruction from the responsible medical center to the local center, for treating the patient if the patient needs a treatment.

22. The method of claim 21, further comprising step of receiving the locality of the local center at the responsible medical center, based on an electronic map system.

23. The method of claim 21, wherein the authorized communication connection is in a wireless manner or a wire manner.
